(19) United States
(12) Patent Application Publication
(10) Pub. No.: US 2008/0075627 A1
(43) Pub. Date: Mar. 27, 2008

(54) Inactivating Biological Agents Dispersed In Gaseous Medium With A Photoactivated Semiconductor

(75) Inventors: Francois Garin, Schiltigheim (FR); Nicolas Keller, Strasbourg (FR); Marie-Claire Lett, Schiltigheim (DE); Valerie Keller-Spitzer, Oberscheeffelsheim (FR); Marc Ledoux, Strasbourg (FR)

Correspondence Address: YOUNG & THOMPSON, 745 SOUTH 23RD STREET, 2ND FLOOR, ARLINGTON, VA, 22202, US

(73) Assignees: Centre National De La Recherche Scientifique (C. N. R. S. ), Paris (FR); Universite Louis Pasteur De Strasbourg, Strasbourg Cedex (FR)

(21) Appl. No.: 11/792,561

(22) PCT Filed: Dec. 7, 2005
(86) PCT No.: PCT/FR05/03074

§ 371 Date: Aug. 7, 2007

(30) Foreign Application Priority Data
Dec. 9, 2004 (FR) 0413152

(51) Int. Cl. A61L 9/18; B01J 19/12

(52) U.S. Cl. 422/4; 422/186

(57) ABSTRACT

A method for inactivating biological agents dispersed within a gaseous medium includes a step which consists in treating the gas flow with a photoactivated semiconductor, in particular with photoactivated titanium oxide. The invention also concerns a device for implementing the method, as well as the method for decontaminating gaseous medium containing harmful, toxic or pathogenic biological agents, such as bacteria.

The present invention relates to decontamination of gas media comprising biological species, and especially to processing of air contaminated with bacteria or viruses.

A number of fields involve gas flows conveying, in a suspended state, biological species which may be found to be harmful or toxic in the short or long term. Examples may especially include in particular the air in climate control systems or air from aerorefrigerated towers which contain fine droplets of water in suspension (aerosols) which may contain harmful bacteria such as those of the genus Legionella, such as Legionella pneumophilia which is responsible for Legionellosis. Another example of contaminated flow of gas is air circulating in hospitals, which is capable of conveying viruses or bacteria which can lead to nosocomial infections. More generally, the air present in any environment which is confined, or which has a high population density is capable of conveying, in a greater or lesser quantity, biological species of the bacterium, virus or spore type which it may be desirable to eliminate.

Currently in order to carry out the decontamination of such vitiated gas media, most of the solutions proposed are relatively expensive and/or complex to carry out and, additionally, their effectiveness is not always sufficient.

In this context, there have been proposed systems for filtering the gas flows. Such filtration systems generally involve high costs, especially since the biological species which it is sought to trap are generally very small. The filtration systems further have the disadvantage of having to be replaced relatively often.

In addition to filtration systems, there have also been proposed other methods for processing gas media, such as, for example, thermal processing, or processing with chemical disinfectants. However, these methods are disadvantageous in that they generally imply that the affected zones become unavailable during the processing operation. Furthermore, some biological species (especially some strains of Legionella pneumophilia) are found to be resistant to that type of thermal or chemical processing operation.

More recently, there have been described, especially in patent applications WO 97/09703 and EP 978690, methods for destroying micro-organisms in contact with photocatalysts activated under UV.

An object of the present invention is to provide a method for processing the above-mentioned gas flows that is of low cost, easy to carry out and at least as effective as, and preferably more effective than, the currently known decontamination methods.

To this end, according to a first aspect, the present invention relates to a method for inactivating biological agents which are dispersed in a gas medium, said process comprising a step consisting in placing the gas medium in contact with a photoactivated semi-conductor material inside a reactor whose internal surface comprises a plurality of protrusions which are arranged in the manner of a helix around a line, the photoactivated semiconductor material being used in the form of a deposit on the internal surface of the reactor, at least on the protrusions. Preferably, the photoactivated semiconductor material used to this end is a photoactivated titanium oxide.

According to a particularly advantageous embodiment, the method of the invention is carried out in a device which comprises:
(i) said reactor, this reactor comprising:
   an internal surface delimiting a passage which extends along a line, said internal surface comprising a plurality of protrusions which are arranged in the manner of a helix around the line,
   an inlet which is suitable for introducing the gas medium containing the dispersed biological agents in the passage, and
   an outlet which is suitable for discharging the medium after the processing operation, the reactor comprising the deposit of semiconductor material deposited on the protrusions of its internal surface, and
(ii) means for irradiating the deposit of semiconductor material, which means photoactivate the semiconductor material of the deposit in the presence of the gas medium containing the biological agents.

The inventors have now evidenced that the deposit of a semiconductor material of the photoactivated TiO₂ type on protrusions of the internal surface of a reactor which are arranged in the manner of a helix allow the biological agents to be inactivated in a specifically effective manner.

The presence of protrusions on the internal surface of the reactor has, inter alia, the advantage of increasing the internal surface-area of the reactor, which allows to increase the exchange surface between the titanium oxide type semiconductor material and the biological agents of the gas medium.

The protrusions further allow a greater quantity of titanium oxide type semiconductor material to be deposited on the internal surface of the reactor. Thus, for example, when the deposit of semiconductor material is carried out by depositing a dispersion of particles of the material on the internal surface of the reactor, then drying (which is often the case in practice), the presence of protrusions brings about a surface “roughness” which allows an increase in the quantity of titanium oxide type semiconductor material which can be deposited on the internal surface of the reactor.

Furthermore, the presence of the above-mentioned protrusions allows the whole of the gas flow to be placed in contact with the titanium oxide present on the internal surface of the reactor, especially by bringing about a turbulent state (or at least a non-laminar state) inside the reactor, which further increases the probability of the biological agents and the photoactivated titanium oxide being brought together.

Besides, the specific arrangement of the protrusions in the form of a helix allows to limit the friction loss within the reactor whilst maintaining high levels of probability of contact between the biological agents of the gas flow and the photoactivated titanium oxide. In fact, the inventors have now evidenced that this arrangement of the protrusions on the internal surface of the reactor in the manner of a helix around a line leads to an ideal compromise between a lower friction loss and a high probability of contact between the biological agents and the photoactivated semiconductor material.

In the present description the term “biological agents” is intended to refer to entities of a biological nature, generally of small size, typically between 0.05 μm (micrometers) and 10 μm (micrometers) and capable of being able to be conveyed by a gas current. Thus, the biological agents to be inactivated according to the method of the invention may especially be bacteria (bacteria of the genus Legionella, such as Legionella pneumophilia, for example), viruses, spores, fungi or a mixture of such entities.

The term “inactivated biological agent”, according to the present description, refers to an agent of the above-mentioned type which has lost a biological activity, and especially which has lost its capacity to replicate (or to reproduce).

Especially, according to the present description, “inactivated bacterium” is intended to refer to a bacterium which is incapable of developing a colony after culture in a suitable medium. Thus, the followings are for example considered to be “inactivated bacteria”:
“dead” bacteria (typically bacteria in which no respiration phenomena are detected); and
bacteria which, though alive, do not develop after culture.

Generally, the method of the invention is carried out on a gas medium comprising biological agents which are harmful, toxic or pathogenic. If necessary, the inactivation most often involves stripping the agents of their harmful, toxic or pathogenic character, especially by inhibiting their capacity to replicate (reproduce).

The “gas medium” in which the above-mentioned biological agents are dispersed is generally air, but it may optionally be another gas which is contaminated with biological agents. Especially in order to obtain adequate efficiency for the decontamination method, however, it is preferable for the gas medium to contain oxygen.

According to a specific embodiment, the gas medium comprising the biological species in the dispersed state is in the form of an aerosol which comprises fine liquid droplets, in general water droplets, which are dispersed within the gas medium, the biological agents being present, entirely or partially, within those droplets.

Thus, for example, the gas medium processed according to the method of the invention may be an aerosol comprising air as a dispersing gas medium and containing water droplets including bacteria and/or viruses, for example, bacteria of the type of the Legionella genus, such as Legionella pneumophilia.

According to another embodiment, the biological species are simply dispersed in the non-modified state within the gas medium. According to this embodiment, the gas medium processed may, for example, be air containing viruses, spores and/or fungi in suspension, those species optionally being deposited on specific supports, such as dust or particles of sand.

Whatever the nature of the biological agents and the gas medium, the inactivation method of the present invention is carried out by placing the gas medium containing the biological agents in contact with a photoactivated semiconductor material, this material preferably being photoactivated titanium oxide.

According to the present invention, the term “semiconductor material” is intended to refer to a material in which the electron states have a band spectrum comprising a valency band and a conduction band which are separated by a forbidden band and in which the energy necessary for causing an electron to pass from the valency band to the conduction band is preferably between 1.5 eV and 4 eV. Such semiconductor materials may in particular include titanium oxide or other metal oxides, such as WO₃, ZnO or SnO₂ or metal sulphides, such as CdS, ZnS or WS₂ or other compounds, such as GaAs, GaP, CdSe or SiC. According to the present invention, titanium oxide is preferably used and leads to particularly satisfactory results.

According to the present description, the term “photoactivated semiconductor material” refers to a semiconductor material of the above-mentioned type which has been subjected to radiation comprising photons having energy greater than or equal to the energy necessary to promote the electrons from the valency band to the conduction band (energy referred to as the “gap” between the valency and conduction bands).

Especially, according to the present description, “photoactivated titanium oxide” is therefore intended to refer to a titanium oxide which has been subjected to radiation comprising photons having energy greater than or equal to the energy necessary to promote the electrons from the valency band to the conduction band, typically radiation containing photons having energy greater than 3 eV, preferably 3.2 eV, and in particular radiation comprising wavelengths less than or equal to 400 nm, for example, less than or equal to 380 nm. Such types of radiation may in particular include the types of radiation provided by ultraviolet radiation lamps of the type referred to as “black light” lamps.

It is known that, in a photoactivated semiconductor material, an in particular in a photoactivated titanium oxide, there are produced, under the effect of radiation of the above-mentioned type, pairs of electrons/holes (a “hole” being an electron deficit in the valency layer left when an electron “jumps” to the conduction band), which confers on the photoactivated semiconductor material pronounced oxidoreduction properties. Those oxidoreduction properties are particularly pronounced in the case of photoactivated titanium oxide and are advantageously used in a number of photocatalytic applications of titanium oxide.

The inventors have now evidenced that a photoactivated semiconductor material such as a photoactivated titanium oxide is in fact found to be sufficiently active to allow the inactivation of biological agents in a gas medium in which the biological agents are, however, greatly dispersed. In this context, it is particularly surprising to find that the method of the invention allows, for example, very dilute gas media to be processed effectively, that is to say, those comprising less than 10⁻³ biological agents per cm³, or less than 10⁻⁴ biological agents per cm³. The activity of a photoactivated semiconductor material of the photoactivated titanium oxide type is further found to be sufficient to effectively process gas media having high contents of biological agents, for example, gas media containing more than 1 biological agent per cm³, and even more than 10 biological agents per cm³ in most cases, even with high rates of gas flow, for example, in the order of from 1 to 10 litres per minute. Thus, the method of the invention generally allows effective processing of gas media typically containing between 10⁻⁴ and 10 biological agents per cm³, for example, between 5.10⁻³ and 5 biological agents per cm³, and in particular dilute media containing between 10⁻⁴ and 0.1 biological agents per cm³ or concentrated media containing between 0.1 and 10 biological agents per cm³.

Without being bound by any particular theory, the results of the work of the inventors seems to suggest that the inactivation of the biological agents is made possible by the highly oxidising character of the photoactivated semiconductor material which appears to bring about photocatalytic degradation of the biological agents which is initiated when those agents come into contact with the surface of the photoactivated semiconductor, and which then continues without having to maintain contact between the semiconductor and the biological agent to be inactivated, which makes it possible to effectively process a medium which is as dilute as a gas dispersion such as an aerosol. Those phenomena are quite particularly marked when the semiconductor used is titanium oxide.

In the present invention, in order to further increase the efficiency of the above-mentioned oxidation mechanisms, it may be advantageous to use, together with the photoactivated semiconductor material, other materials which have an oxidising character. In this context, it is in particular possible to use materials based on a semiconductor, such as titanium oxide and further containing metals, such as gold or silver in the metal form, for example, in the form of particles dispersed in the semiconductor material or deposited on its surface.

According to this specific embodiment, the mass ratio (additional oxidant/semiconductor) advantageously remains less than 5%, or 3%, and it is typically between 0.5 and 2%. The presence of such additional oxidants is not generally necessary, however, in order to obtain effective processing.

Furthermore, the photocatalytic degradation of biological agents which was observed by the inventors has the advantage of not being very selective, which means that any biological agent is in principle capable of being degraded by being placed in contact with a photoactivated semiconductor of the photoactivated titanium oxide type. In practice, it has been found that photoactivated titanium oxide effectively has a very wide decontamination spectrum.

Another advantage of the method is that the particularly effective photocatalytic degradation which is observed with a photoactivated semiconductor of the photoactivated titanium oxide type is obtained very simply and cheaply in that it only requires irradation with types of radiation having relatively low energy. Thus, in the case of titanium oxide, for example, only radiation energy levels in the order of from 3 to 3.2 eV, that is to say, wavelengths in the order of from 380 to 400 nm, are required. The energy required for photoactivation can further be reduced if the semiconductor material is doped (for example, with metals such as chromium or compounds based on N, S or C) or using chromophoric agents (for example, anthracenes or anthracines) in association with the semiconductor material, In that case, very low activation energy levels may be sufficient to photoactivate the material and may correspond, for example, to wavelengths greater than or equal to 500 nm, for example, greater than or equal to 550 nm.

The irradiation of the titanium oxide is generally carried out under radiation containing radiation levels of the near ultraviolet range, for example, by irradiation with sunlight or sodium vapour lamps or so-called “black light” lamps, which are radiation levels which it is possible to obtain at low cost. Furthermore, it should be noted that the radiation used for photoactivating the TiO₂, or more generally the semiconductor, is generally radiation having insufficient energy to bring about alone inactivation of the biological agents in the absence of any semiconductor of the TiO₂ type.

In other words, the levels of radiation used to photoactivate the semiconductor in the method of the invention are not generally per se radiation levels having sufficient energy to bring about a germicidal effect. The levels of radiation used to photoactivate the semiconductor materials according to the method of the invention thus generally have wavelengths greater than 254 nm, and typically greater than 320 nm, for example, greater than or equal to 350 nm.

It should further be emphasised that no heating is required to carry out the photoactivation of the titanium oxide, which allows the method of the invention to be carried out at ambient temperature, for example, between 10 and 30° C.

The precise nature of the semiconductor material used according to the invention is not generally decisive for obtaining the desired effect of inactivating the biological agents.

Thus, in the case of titanium oxide, for example, any commercial titanium oxide may be used effectively in the method of the invention, which further constitutes an advantage of the method.

Nevertheless, according to an embodiment leading to good results in terms of inactivating the biological agents, the titanium oxide used according to the method of the invention contains TiO₂ in anatase form, preferably at a ratio of at least 50%. Thus, according to this embodiment, the titanium oxide used may, for example, be mainly constituted (that is to say, generally for at least 99% by mass and preferably for at least 99.5% by mass, or for at least 99.9% by mass) by TiO₂ in anatase form.

The use of TiO₂ in rutile form is also found to be advantageous in that TiO₂ in this form is photoactivated by the spectrum of visible light.

According to another advantageous embodiment, the titanium oxide used comprises a mixture of TiO₂ in anatase form and TiO₂ in rutile form with a proportion of anatase/rutile of preferably between 50/50 and 99/1, for example, between 70/30 and 90/10, and typically in the order of 80/20.

Furthermore, especially in order to optimise the exchanges between the semiconductor material of the titanium oxide type and the biological agents dispersed in the gas flow, it is most often advantageous for the semiconductor material used to have a specific surface-area of between 20 and 500 m²/g, preferably greater than or equal to 40 m²/g and even more advantageously at least equal to 100 m²/g and quite particularly when titanium oxide is involved. The specific surface-area to which reference is made here is the specific BET surface-area measured by adsorption of nitrogen according to the technique known as the Brunauer-Emmet-Teller technique. To this end, it is in particular possible to use a titanium oxide which has per se a high specific surface-area or a titanium oxide which is deposited on a porous support (such as, for example, a silica support) having a high specific surface-area.

A particularly advantageous titanium oxide useful in the method of the invention is the titanium oxide marketed by the company Degussa under the name TiO₂ of the type P25.

The photoactivated semiconductor material which is used according to the invention may be in various physical forms in accordance with the gas medium processed and in particular in accordance with the volume of this medium and the rate at which it is desirable to carry out the processing operation. Generally, the titanium oxide type semiconductor material can be used in any form suitable for being irradiated with radiation having a wavelength allowing its photoactivation and allowing the titanium oxide in the photoactivated state to be brought into contact with the biological agents to be processed, on condition that it is accessible for inactivating the biological agents.

In the method of the invention, the titanium oxide type semiconductor material used is used in the immobilised state on the internal surface of the reactor, the gas medium to be processed being brought into contact with that modified surface. According to a specific method, the surface on which the titanium oxide type semiconductor material is immobilised may be a surface on which a support having a high specific surface-area (for example, a layer of silica) is deposited, the semiconductor material being immobilised on this support. According to another embodiment, the titanium oxide type semiconductor material can be used in the form of a deposit obtained by depositing a film of a dispersion (for example, an aqueous dispersion) of particles based on the titanium oxide type semiconductor on a surface and by then drying the film obtained.

According to another specific aspect, the present invention also relates to a device suitable for carrying out the above-mentioned method for inactivating biological agents dispersed within a gas medium.

This device comprises a reactor including:
an internal surface delimiting a passage which extends along a line, the internal surface comprising a plurality of protrusions arranged in the manner of a helix around said line;
an inlet which is suitable for introducing the gas medium containing the dispersed biological agents in the passage; and
an outlet which is suitable for discharging the gas medium after inactivating the biological agents.

The reactor further comprises, as a means for inactivating biological agents dispersed in a gas medium, a deposit of semiconductor material (preferably a deposit of titanium oxide) which is immobilised on the protrusions from its internal surface, associated with means for irradiating the deposit of titanium oxide capable of photoactivating the titanium oxide of the deposit in the presence of the gas medium containing the biological agents.

The deposit based on a semiconductor material which is present on the internal surface of the reactor is preferably a deposit of titanium oxide selected from the preferred titanium oxides mentioned above. Thus, it is advantageously a titanium oxide containing TiO₂ in anatase form or a mixture of rutile/anatase and having a specific surface-area of between 20 and 500 m²/g. This deposit can be obtained, for example, by depositing a film of a dispersion (for example, an aqueous dispersion) of particles based on a titanium oxide type semiconductor material on the internal surface of the reactor, then by drying the film obtained. This deposit can also be obtained by drying a film of a dispersion in a non-aqueous solvent in which the semiconductor used is not soluble. When the photoactivated semiconductor used is titanium oxide, a deposit of titanium oxide on the surface may be carried out by depositing a solution of titanate and by thermally processing the deposit obtained in this manner, by means of which the formation of TiO₂ is obtained from the titanate precursor.

The deposit of titanium oxide type semiconductor material may be of the continuous or discontinuous type and it is preferably a continuous solid film which is distributed over the whole of the internal surface of the reactor, in particular in order to optimise the exchange surface between the gas flow and the photoactivated titanium oxide. This deposit preferably further has a mean thickness of between 0.5 μm and 100 μm, for example, between 1 and 20 μm, this thickness typically being in the order of 5 μm.

The irradiation means associated with this deposit of a titanium oxide type semiconductor are generally radiation sources comprising photons having energy greater than 3 eV (preferably greater than 3.2 eV), for example, one or more lamps emitting radiation types comprising wavelengths of less than 300 nm (for example, less than 400 nm), for example, lamps of the type of black light lamps or visible light lamps. According to a specific embodiment, the source of radiation used may be sunlight. In general, those radiation sources are located outside the reactor. If necessary, in order to allow activation of the titanium oxide, the wall of the reactor is constituted by a material that is transparent to at least a portion of the effective radiation emitted by the sources, that is to say that the wall of the reactor allows at least a portion of the types of radiation which have sufficient energy to activate the titanium oxide to pass. To that end, reactors composed of glass, in particular pyrex, are preferably used in general.

The device of the invention allows the biological agents present in the gas flow to be inactivated effectively. The reactor used further has the advantage of being able to be used in all positions (that is to say, horizontal, vertical or inclined), in particular taking into consideration the immobilisation of the titanium oxide on the walls of the reactor. Furthermore, its inlet and outlet can be transposed which allows, if necessary, the direction of the gas flow being processed to be inverted.

In particular in accordance with the quantity of gas medium to be processed and the envisaged flow rate, different dimensions and geometries may be envisaged for the reactor.

The protrusions present on the internal surface of the reactor are advantageously integral with the wall of the reactor. Such protrusions are especially easy to obtain when a reactor composed of pyrex is used: in this context, the protrusions can be constructed by hot-forming the wall of the reactor.

The number and the geometry of the protrusions may vary to quite a large degree, especially by taken into account the desired application and rate of the gas flow being processed. Naturally, for a given projection geometry, the probability of contact between the biological agents and the photoactivated titanium oxide increases with the number of protrusions. That being the case, at the same time, the presence of protrusions can bring about friction loss in the reactor which become increasingly large as the number of protrusions increases, in particular in the case of reactors having large dimensions.

In order to limit the friction loss of this type whilst still maintaining high levels of probability of contact between the biological agents of the gas flow and the photoactivated titanium oxide, besides the arrangement of the protrusions in the manner of a helix, it is also possible to modify the geometry of the protrusions and their arrangement over the internal surface of the reactor. In this context, it should be noted that the protrusions may have surfaces which are orientated in a co-current or counter-current manner. If it is desirable to limit the friction loss, it is preferable to select protrusions having surfaces orientated in a co-current manner. In this context, the protrusions may advantageously be of conical shape, which allows, if necessary, the direction of the gas flow to be inverted in the reactor whilst maintaining the protrusions with co-current orientation.

The method and the device of the invention will now be described in greater detail in the following description, given with reference to the appended drawings in which:

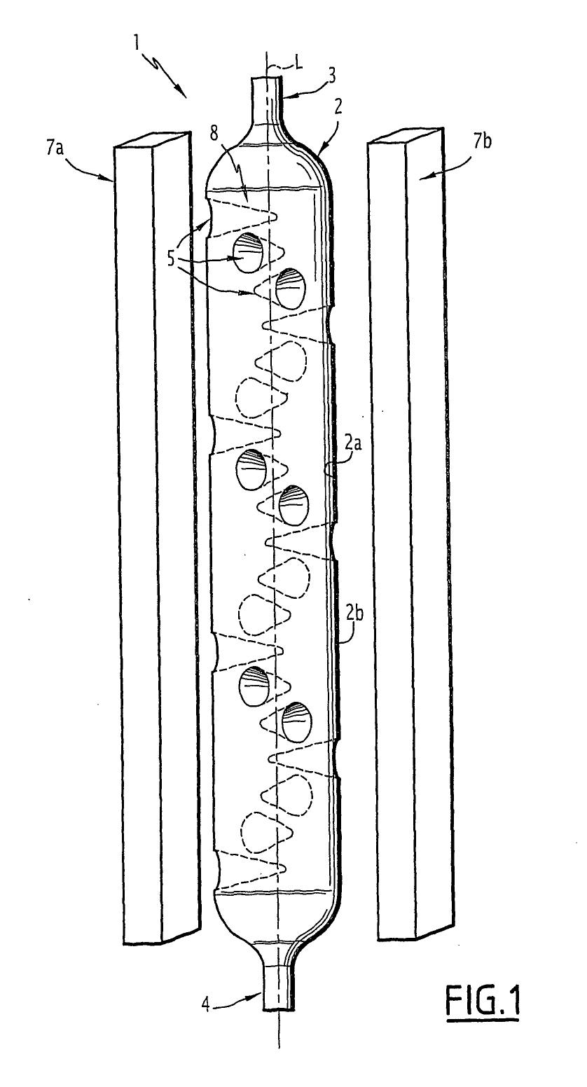

FIG. 1 is a schematic side elevation of a device in accordance with a particularly advantageous embodiment of the invention; and

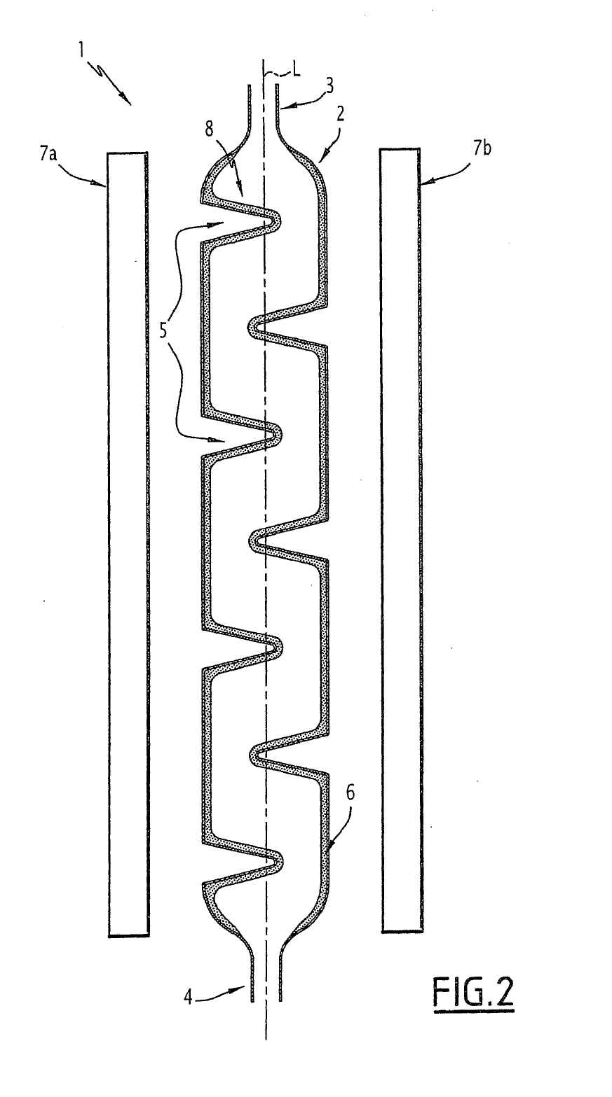

FIG. 2 is a schematic sectional view of the reactor of FIG. 1.

The device 1 of the appended Figures comprises a tubular reactor 2 which is constituted by a pyrex tube constricted at its two ends in order to form an inlet 3 and an outlet 4 which are connected to means for conveying the gas flow to be processed (not illustrated). The reactor 2 has an internal surface 2a which defines, in the reactor 2, a passage which extends along a line L between the inlet 3 and the outlet 4. The reactor 2 comprises a series of protrusions 5 which are advantageously (but not necessarily) substantially conical, and which are integral with the internal surface 2a of the reactor 2 and extend radially towards the inner side of the reactor 2. Those protrusions 5 may be obtained, for example, by hot-forming the pyrex tube by means of a punch which is applied to the external surface 2b of the reactor. The protrusions 5 are indented at the external surface 2b. The protrusions 5 are arranged along a helical line at the internal surface of the reactor, as is visible in FIG. 1. In other words, the protrusions are arranged in a helical manner around the line L.

The reactor 2 comprises, at its internal surface, a deposit of semiconductor material, preferably a deposit of titanium oxide 6 (not illustrated in FIG. 1), which may be obtained by depositing a film of an aqueous dispersion of titanium oxide type semiconductor particles on the internal surface of the reactor, then drying the film obtained and optionally repeating those operations. The film typically has a thickness in the order of 5 μm (micrometres), that thickness being able to be modified in particular by influencing the initial concentration of the dispersion of titanium oxide particles and the number of depositing/drying cycles. The deposit 6 particularly covers the protrusions 5.

In addition to the reactor 2, the device 1 further comprises lamps 7a and 7b which emit types of radiation comprising, inter alia, wavelengths which are less than or equal to 400 nm and preferably less than or equal to 380 nm (typically the lamps 7a and 7b may be UV emission lamps (of the “black light” type) or visible light emission lamps which are arranged so as to irradiate the whole of the titanium oxide deposit 6). In the Figures, only two lamps are illustrated but in practice the number of lamps and their power may vary. By way of example, for a cylindrical reactor which has a diameter of 6 cm and a length of 30 cm, 4 lamps having a power of 8 W of the black light tube type (for example, of the type marketed by the company Philips under the name TL8W-08) distributed around the reactor are generally sufficient.

When the device 1 is used to carry out the inactivation method of the invention, a gas flow comprising biological agents in suspension is introduced into the reactor via the inlet 3. To this end, the device generally comprises, upstream of the inlet 3, injection means which are not illustrated in the Figures. In addition to those injection means, it may be advantageous for the device to comprise means for branching a portion of the incoming gas flow which are associated with qualitative and/or quantitative analysis means for the biological agents in the gas flow, those analysis means comprising, for example, membranes which allow the biological agents to be collected.

The gas flow introduced into the reactor then comes into contact with the oxide deposit 6 which is present on the internal wall of the reactor and which is photoactivated under the effect of the radiation provided by the lamps 7a and 7b.

The occurrences of contact between the biological agents and the photoactivated titanium oxide mostly take place in the region of the upper surfaces 8 of the protrusions 5. Taking into account the substantially conical shape of those protrusions, the upper surfaces 8 of the protrusions are orientated in a co-current manner and thereby limit the friction loss. It should be noted that the reactor has excellent symmetry and that it can be used with the direction of the current being inverted (that is to say, by transposing the inlet and the outlet), retaining the same advantages.

The arrangement of the protrusions 5 in a helical manner is also so as to limit the friction loss within the reactor, quite particularly for reactors having large dimensions. That specific organisation of the protrusions further brings about a convection movement of the gas flow which further brings about agitation which allows the various biological agents present in suspension to be brought into contact with the photoactivated deposit 6.

Taking into account those conditions, the device 1 can be used with gas flow rates which are typically between 1 and 10 litres per minute, with good efficiency concerning the process for inactivating the biological agents.

Thus, at the outlet 4 of the reactor, a gas flow is generally collected in which the greater portion of the biological agents are inactivated.

The device 1 may comprise, downstream of the outlet 4, means for branching a portion of the gas flow being discharged, which means are associated with qualitative and/or quantitative analysis means for the biological agents in the gas flow, similar to those which may be present upstream of the inlet 3.

The method of the invention, especially when it is carried out by means of a device as described in detail above, has been found to be particularly effective for decontaminating gas media containing biological agents which are harmful, toxic or pathogenic.

According to another aspect, this use constitutes another object of the present invention.

Various features and advantages of the invention will be further appreciated from the illustrative example set out below.

### EXAMPLE

Various aerosols comprising Escherichia Coli bacteria were processed by means of a device as described in the appended FIGS. 1 and 2 having the following characteristics:
diameter of the reactor: 6 cm
length of the reactor: 30 cm
titanium oxide used: TiO₂ of the type P25 marketed by the company Degussa (titanium oxide having a ratio of the rutile/anatase forms of 20/80) present in the reactor in the form of a deposit having a mean thickness of approximately 5 μm and having a BET of 50 m²/g
lamps used: 4 UV radiation lamps (black light tube) of the TL8W-08 type which are marketed by the company Philips, each having a power of 8 W, and which are orientated towards the reactor and arranged around the reactor at a distance of 3 cm from the walls.

Two types of aerosols were formed based on two aqueous suspensions of Escherichia Coli comprising 10⁷ and 10⁸ CFU per litre (CFU=Colony Forming Unit). These aerosols were produced by injecting compressed air at a pressure of 1.5 bar (1.5.10⁻⁵ Pa) into each of the aqueous suspensions of Escherichia Coli bacteria using a Laskin type nozzle and an aerosol generator of the PLG2000 type marketed by the company PALAS.

In each case, the content of bacteria forming colonies in the aerosol at the inlet and at the outlet of the reactor was quantified. That quantification was carried out by incubating on nutrient gelose the filtered bacteria which are obtained by filtering the incoming flow and the discharge flow of the reactor in the permanent state for a period of 5 minutes over a membrane filter of cellulose ester having a pore diameter of 0.45 μm (millipore).

The conditions of the tests carried out and the results obtained are set out in Tables I and II below.

TABLE I
Inactivation of aerosols containing Escherichia Coli
bacteria (concentration of Escherichia Coli of
10⁸ CFU in the initial suspension)

| | | Test | | |
|---|---|---|---|---|
| | | 1 | 2 | 3 |
| Flow rate of aerosol | | 90 L/h | 240 L/h | 360 L/h |
| in the reactor | | (1.5 L/min) | (4 L/min) | (6 L/min) |
| Bacteria | At the inlet | 200 | 310 | 410 |
| content | of the reactor | | | |
| forming a | At the outlet | 0 | 1 | 1 |
| colony (CFU) | of the reactor | | | |

TABLE II
Inactivation of aerosols containing Escherichia Coli
bacteria (concentration of Escherichia Coli of
10⁷ CFU in the initial suspension)

| | | Test | | |
|---|---|---|---|---|
| | | 1 | 2 | 3 |
| Flow rate of aerosol | | 90 L/h | 240 L/h | 360 L/h |
| in the reactor | | (1.5 L/min) | (4 L/min) | (6 L/min) |
| Bacteria | At the inlet | 110 | 160 | 220 |
| content | of the reactor | | | |
| forming a | At the outlet | 0 | 0 | 0 |
| colony (CFU) | of the reactor | | | |

In each case, the filtered bacteria at the inlet and outlet of the reactor were further examined by epifluorescence microscopy using 5-cyano-2,3-ditolyl tetrazolium chloride so as to identify the bacteria which are breathing (which appear red) and the others. In that case, the filters which were used are polycarbonate membrane filters. In all cases, a population of bacteria was observed at the inlet of the reactor, most of which appeared with the red coloration that is characteristic of respiration phenomena. Conversely, at the outlet of the reactor, the majority of the bacteria observed no longer have that red coloration.

1-15. (canceled)

16. A method for inactivating biological agents which are dispersed in a gas medium, said method comprising a step consisting in placing the gas medium in contact with a photoactivated semiconductor material inside a reactor (2) whose internal surface comprises a plurality of protrusions (5) which are arranged in a helical manner around a line (L), the photoactivated semiconductor material being used in the form of a deposit (6) on the internal surface of the reactor, said deposit being at least on the protrusions (5).

17. The method of claim 16, wherein the used photoactivated semiconductor material is a photoactivated titanium oxide.

18. The method of claim 16, said method being carried out in a device (1) which comprises:
said reactor (2), this reactor comprising an internal surface delimiting a passage which extends along a line (L), the internal surface comprising a plurality of protrusions (5) which are arranged in the manner of a helix around the line (L), an inlet (3) which is suitable for introducing the gas medium containing the dispersed biological agents in the passage, and an outlet (4) which is suitable for discharging the medium after the processing operation, the reactor comprising the deposit of semiconductor material (6) deposited on the protrusions (5) of its internal surface, and
means (7a, 7b) for irradiating the deposit of semiconductor material, which means photoactivate the semiconductor material of the deposit (6) in the presence of the gas medium containing the biological agents.

19. The method of claim 16, wherein the biological agents dispersed in the gas medium are bacteria.

20. The method of claim 16, wherein the gas medium comprising the biological species in the dispersed state is in the form of an aerosol which comprises fine liquid droplets which are dispersed within the gas medium, the biological agents being present, entirely or partially, within those droplets.

21. The method of claim 20, wherein the gas medium is an aerosol comprising air as a dispersing gas medium and containing water droplets including bacteria and/or viruses.

22. The method of claim 21, wherein the droplets include bacteria of the Legionella genus, such as Legionella pneumophilia.

23. The method of claim 22, wherein the biological agents are present in the gas medium at a content of between 10⁻⁴ and 10 biological agents per cm³.

24. The method of claim 23, wherein the photoactivated titanium oxide used is a titanium oxide subjected to radiation comprising wavelengths of between 254 and 400 nm.

25. The method of claim 16, wherein the semiconductor material used is a titanium oxide containing TiO₂ in anatase form.

26. The method of claim 25, wherein the used titanium oxide comprises a mixture of TiO₂ in anatase form and TiO₂ in rutile form with a proportion of anatase/rutile of between 50/50 and 99/1.

27. The method of claim 16, wherein the semiconductor material used has a specific surface-area of between 20 and 500 m²/g.

28. A device (1) for carrying out a method for inactivating biological agents dispersed within a gas medium according to claim 16, said device comprising
a reactor (2) comprising an internal surface delimiting a passage which extends along a line (L), the internal surface comprising a plurality of protrusions (5) arranged in the manner of a helix about the line (L), an inlet (3) which is suitable for introducing the gas medium containing the dispersed biological agents in the passage, and an outlet (4) which is suitable for discharging the medium after the processing operation, the reactor comprising, as a means for inactivating biological agents dispersed in a gas medium, a deposit of semiconductor material (6) which is deposited on the protrusions (5) from its internal surface, and
means (7a, 7b) for irradiating the deposit of semiconductor material, which means are capable of photoactivating the semiconductor material of the deposit (6) in the presence of the gas medium containing the biological agents.

29. The device (1) of claim 28, wherein the deposit of semiconductor material (6) is a titanium oxide deposit.

30. The method of claim 16, which is used for decontaminating a gas medium containing biological agents which are harmful, toxic or pathogenic.

31. A device (1) for carrying out a method for inactivating biological agents dispersed within a gas medium according to claim 17, said device comprising
a reactor (2) comprising an internal surface delimiting a passage which extends along a line (L), the internal surface comprising a plurality of protrusions (5) arranged in the manner of a helix about the line (L), an inlet (3) which is suitable for introducing the gas medium containing the dispersed biological agents in the passage, and an outlet (4) which is suitable for discharging the medium after the processing operation, the reactor comprising, as a means for inactivating biological agents dispersed in a gas medium, a deposit of semiconductor material (6) which is deposited on the protrusions (5) from its internal surface, and
means (7a, 7b) for irradiating the deposit of semiconductor material, which means are capable of photoactivating the semiconductor material of the deposit (6) in the presence of the gas medium containing the biological agents.

32. A device (1) for carrying out a method for inactivating biological agents dispersed within a gas medium according to claim 18, said device comprising
a reactor (2) comprising an internal surface delimiting a passage which extends along a line (L), the internal surface comprising a plurality of protrusions (5) arranged in the manner of a helix about the line (L), an inlet (3) which is suitable for introducing the gas medium containing the dispersed biological agents in the passage, and an outlet (4) which is suitable for discharging the medium after the processing operation, the reactor comprising, as a means for inactivating biological agents dispersed in a gas medium, a deposit of semiconductor material (6) which is deposited on the protrusions (5) from its internal surface, and
means (7a, 7b) for irradiating the deposit of semiconductor material, which means are capable of photoactivating the semiconductor material of the deposit (6) in the presence of the gas medium containing the biological agents.

33. A device (1) for carrying out a method for inactivating biological agents dispersed within a gas medium according to claim 19, said device comprising
a reactor (2) comprising an internal surface delimiting a passage which extends along a line (L), the internal surface comprising a plurality of protrusions (5) arranged in the manner of a helix about the line (L), an inlet (3) which is suitable for introducing the gas medium containing the dispersed biological agents in the passage, and an outlet (4) which is suitable for discharging the medium after the processing operation, the reactor comprising, as a means for inactivating biological agents dispersed in a gas medium, a deposit of semiconductor material (6) which is deposited on the protrusions (5) from its internal surface, and
means (7a, 7b) for irradiating the deposit of semiconductor material, which means are capable of photoactivating the semiconductor material of the deposit (6) in the presence of the gas medium containing the biological agents.

34. A device (1) for carrying out a method for inactivating biological agents dispersed within a gas medium according to claim 20, said device comprising
a reactor (2) comprising an internal surface delimiting a passage which extends along a line (L), the internal surface comprising a plurality of protrusions (5) arranged in the manner of a helix about the line (L), an inlet (3) which is suitable for introducing the gas medium containing the dispersed biological agents in the passage, and an outlet (4) which is suitable for discharging the medium after the processing operation, the reactor comprising, as a means for inactivating biological agents dispersed in a gas medium, a deposit of semiconductor material (6) which is deposited on the protrusions (5) from its internal surface, and
means (7a, 7b) for irradiating the deposit of semiconductor material, which means are capable of photoactivating the semiconductor material of the deposit (6) in the presence of the gas medium containing the biological agents.

35. A device (1) for carrying out a method for inactivating biological agents dispersed within a gas medium according to claim 21, said device comprising
a reactor (2) comprising an internal surface delimiting a passage which extends along a line (L), the internal surface comprising a plurality of protrusions (5) arranged in the manner of a helix about the line (L), an inlet (3) which is suitable for introducing the gas medium containing the dispersed biological agents in the passage, and an outlet (4) which is suitable for discharging the medium after the processing operation, the reactor comprising, as a means for inactivating biological agents dispersed in a gas medium, a deposit of semiconductor material (6) which is deposited on the protrusions (5) from its internal surface, and
means (7a, 7b) for irradiating the deposit of semiconductor material, which means are capable of photoactivating the semiconductor material of the deposit (6) in the presence of the gas medium containing the biological agents.
